(19) United States
(12) Patent Application Publication
(10) Pub. No.: US 2006/0137694 A1
(43) Pub. Date: Jun. 29, 2006

(54) Apparatus and method for providing medical emergency assistance instructions

(76) Inventors: David D. Probert, Clinton, UT (US); Gabriel Chino, Clinton, UT (US)

Correspondence Address: Todd E. Zenger;KIRTON & McCONKIE, 1800 Eagle Gate Tower, 60 East South Temple, Salt Lake City, UT, 84111, US

(21) Appl. No.: 11/021,736

(22) Filed: Dec. 23, 2004

(51) Int. Cl. A61B 19/00; A61M 16/00

(52) U.S. Cl. 128/897; 128/898

(57) ABSTRACT

An electronic device with emergency instructions to instruct a user how to handle a variety of medical as well as non-medical emergencies, and the instructions are electronically resident on the electronic device. The electronic device will generally be a device generally used for many different tasks such as telecommunication or planning. Finally the instructions may be installed during the device's manufacture or after the device's manufacture. The instructions may be accessed at any time and anywhere because they are resident upon the device.

### BACKGROUND

There is a real danger associated with being injured outside established community centers. This is especially true when an injury occurs in an isolated area and no help can be reached through telecommunication. Despite the advent of mobile phones, satellite phones, and other such modern telecommunication means, accident victims do not always have ready access to medical emergency technicians. Thus, when a victim is injured and cannot immediately contact emergency medical personnel, the danger posed by the injury greatly increases.

Many injuries are worsened due to lack of timely or competent emergency medical care. Few civilians are adequately trained in cardiopulmonary resuscitation (CPR) and basic first aid. Currently, members of the public rely on cell phones or other telecommunication means to receive detailed instructions on how to perform basic CPR and other first aid techniques. However, relying on telecommunication devices to receive detailed instructions to perform CPR and other first aid techniques subjects the accident victim to potentially aggravating circumstances. For example, telecommunication services are not available in vast geographic areas. Mobil calls are frequently dropped over the wireless network. Signal quality and strength varies making it difficult to hear instructions received over telecommunication networks. Accordingly, these shortcomings pose challenges and shortfalls for accident victims, especially when the victim is injured outside the zone of available wireless communication.

### BRIEF SUMMARY

The present invention teaches a device and method for providing medical emergency assistance instructions independent of the device's proximity to or connection with a zone of wireless communication network. More specifically, the present invention teaches a device that provides a user a series of interactive menus to identify proper treatments for accident victims. The present invention teaches a method for accessing instructions for proper CPR and other first aid techniques.

### BRIEF DESCRIPTION OF THE DRAWINGS

FIG. 1A illustrates an exemplary schematic representation of the present invention.

FIG. 1B illustrates potential use of the present invention independent of any telecommunications network.

FIGS. 2-20 illustrate flow charts or exemplary interactive menus as taught by the present invention.

### DETAILED DESCRIPTION OF EXEMPLARY EMBODIMENTS

This specification describes exemplary embodiments and applications of the invention. The invention, however, is not limited to these exemplary embodiments and applications or to the manner in which the exemplary embodiments and applications operate or are described herein.

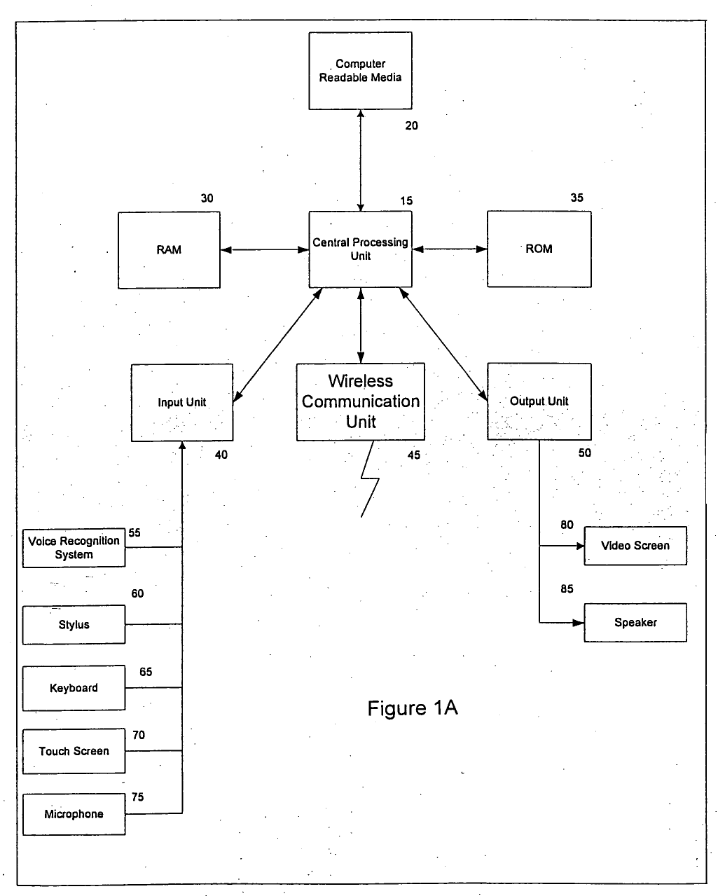

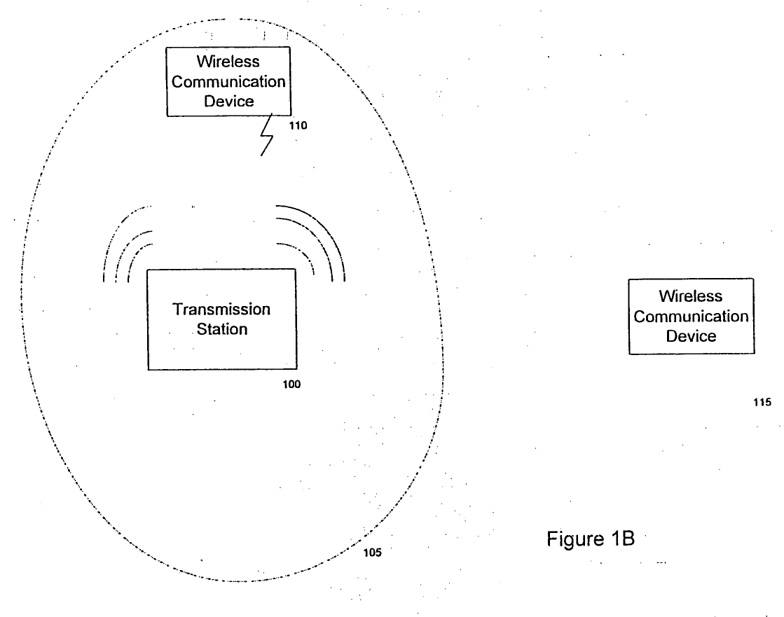

Referring now to FIG. 1A, an exemplary embodiment of the present invention is illustrated. The present invention comprises a an electronic device such as wireless communication device 10 having a central processing unit 15 in communication with a computer readable media 20 such as an optical drive, flash memory, or an internal hard drive.

The present invention contemplates downloading medical emergency assistance programs to device 10 and storing them in a computer-readable media. The program of instructions can be downloaded into the device and stored therein from a compact disc or other computer readable media as is commonly known in the art. However, the present invention also contemplates installing a computer-readable medium into the communication device 10 upon manufacture or assembly such as installing flash memory either upon the device's initial manufacture or downloading or installing the instructions after initial manufacture so as to be electronically resident or stored in the device 10. The instructions may become resident by storing a program containing the instruction on a computer readable media in device 10.

The central processing unit 15 is also in communication with RAM 30 and ROM 35 as is commonly taught in the art.

The central processing unit 15 is also in communication with an input unit 40, an optional wireless communication unit 45, and an output unit 50. The input unit 40 provides a user means by which to input data and interact with interactive menus generated by the program resident or stored on computer-readable media 20. For example, a user may activate the interactive menus using voice recognition system 55 and identify the ailment afflicting an accident victim also through voice recognition technology. Alternatively, a user may use a stylus 60, keyboard 65, a touch-screen 70, or a microphone 75 by which to interact with the interactive menus stored on computer-readable media 20. The input unit 40 may also comprise a dedicated procedure such as a single button or a combination of buttons.

The output unit 50 as taught by the present invention comprises a video screen 80 or speaker 85 by which the user may interact with the interactive menus stored on computer-readable media 20. It is also taught by the present invention that the speaker 85 may be an earpiece linked to the CPU via an alternative wireless means such as infrared technology. Thus one exemplary embodiment of the present invention teaches using a combination of the input unit 40 and the output unit 50 to interact with the interactive menus in order to identify an ailment afflicting an accident victim, and receive the necessary emergency medical instructions needed to help the accident victim. However, an alternative exemplary embodiment of the present invention teaches an electronic device 10 with an output unit 50, such as a speaker or visual display that instructs a user, the device may be activated remotely by a proximate activation device.

Finally, the optional wireless communication unit 45 allows the device as taught in the present invention to communicate via wireless means. However, the present invention teaches that the wireless communication device may be inside or outside the zone of wireless communication and still provide the user access to the interactive menus.

The electronic device 10 as taught in the present invention can be a cell phone, a pocket personal computer, a pager, a walkie-talkie, a global positioning system, a kiosk display, a personal digital assistant, a cordless telephone, a calculator, or any other wireless communication device known in the art. The present invention further teaches that the CPR and first aid instructions can be downloaded over a wireless network or can be installed manually such as using a flash memory or other types of diskettes or memory known in the art.

Additionally, the instruction could be installed by the manufacturer, and the user could update the instructions as desired over a wireless network, or through manually inserting additional memory. Furthermore, the instructions could be tailored to a specific user's medical needs. For example, a user who suffers from sever allergies may download specific instructions on how to treat him or her if an allergic reaction should occur. In addition, instructions for general non-medical emergencies could also be downloaded. For example, how to be found once lost, how to start a fire without matches, or how to change a tire.

Referring now to FIG. 1B, where an exemplary illustration of the present invention is shown, there is a transmission station 100, which could be a cell tower, a relay station, or any other transmission means known in the art. Around the transmission station 100 is a zone of wireless communication 105 wherein a wireless device 110 compatible with the transmission station 100 has wireless communication capabilities that would allow the wireless communication device 110 to either communicate with station 100 or download information from the wireless network. Wireless communication device 110 is within the zone of communication 105 and this proximity to transmission station 100 that a user of the wireless device 110 could download interactive menus.

However, also illustrated in FIG. 1B is wireless communication device 115, which is outside the zone of wireless communication. While the wireless communication device 115 may not be used for telecommunication or for downloading information, the interactive menus are still available to the user because they are stored on the wireless communication device 115. Accordingly, a user's ability to access the interactive menus and gain instruction on how to provide emergency medical assistance to an accident victim is independent of any transmission tower or connectivity to any telecommunications network.

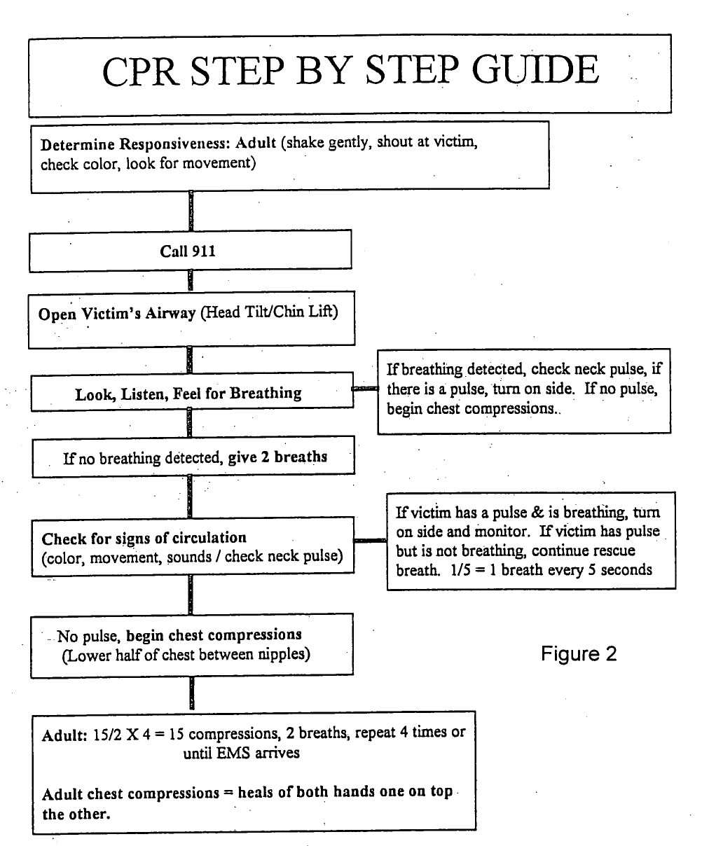

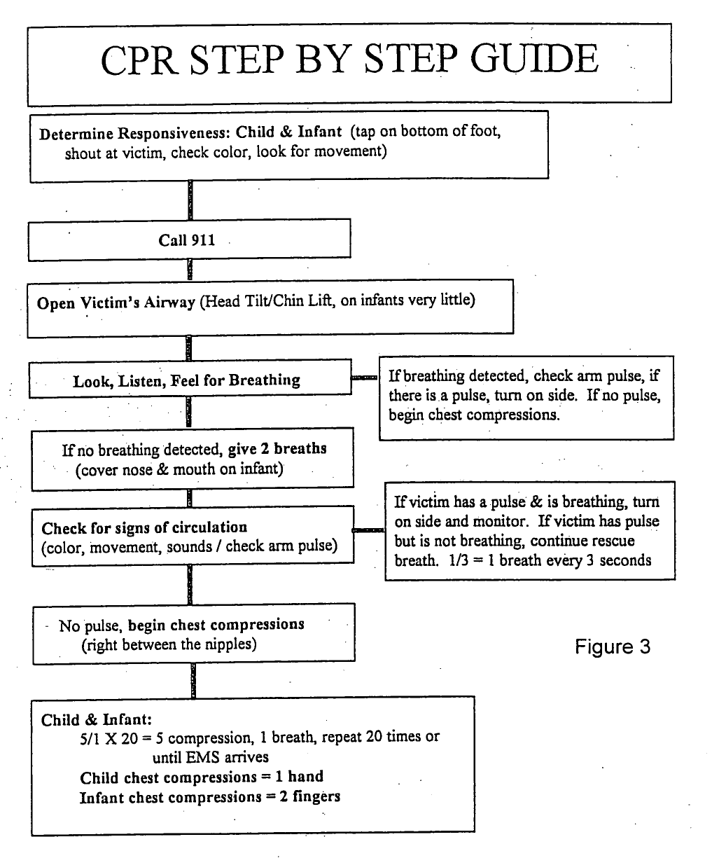

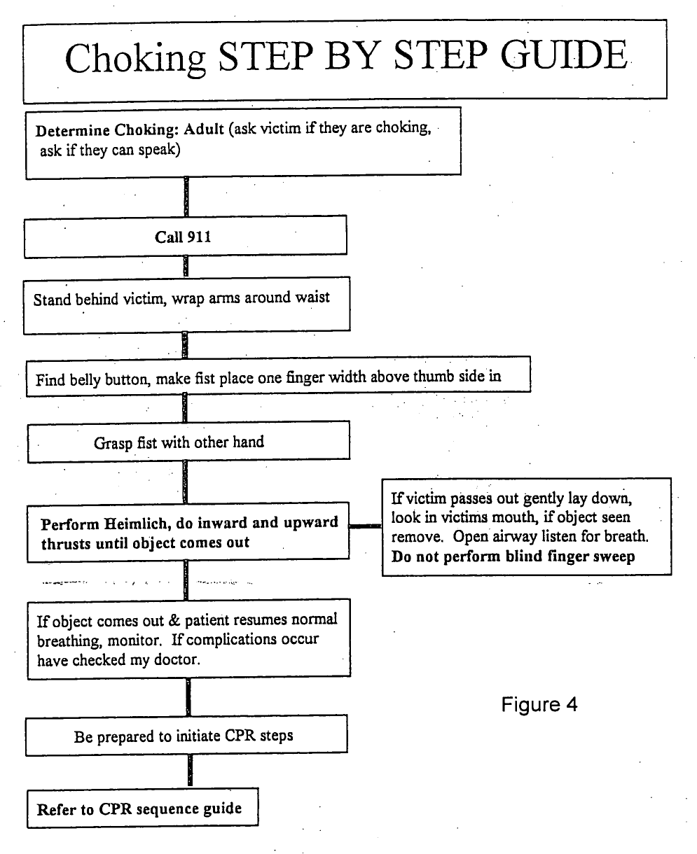

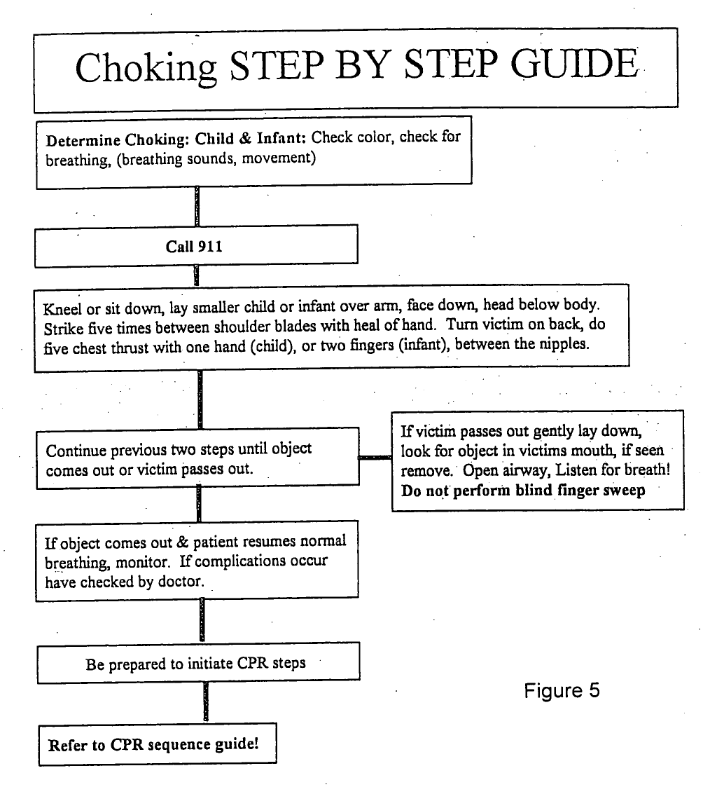

FIGS. 2-20 provide exemplary illustrations of interactive menus contemplated by the present invention. FIG. 2 illustrates an interactive menu for instructions on treating an adult with CPR, while FIG. 3 illustrates emergency medical instructions on how to provide CPR to a child or an infant. FIG. 4, illustrates instructions for treating a choking adult, while FIG. 5 illustrates instructions for treating a choking child or infant.

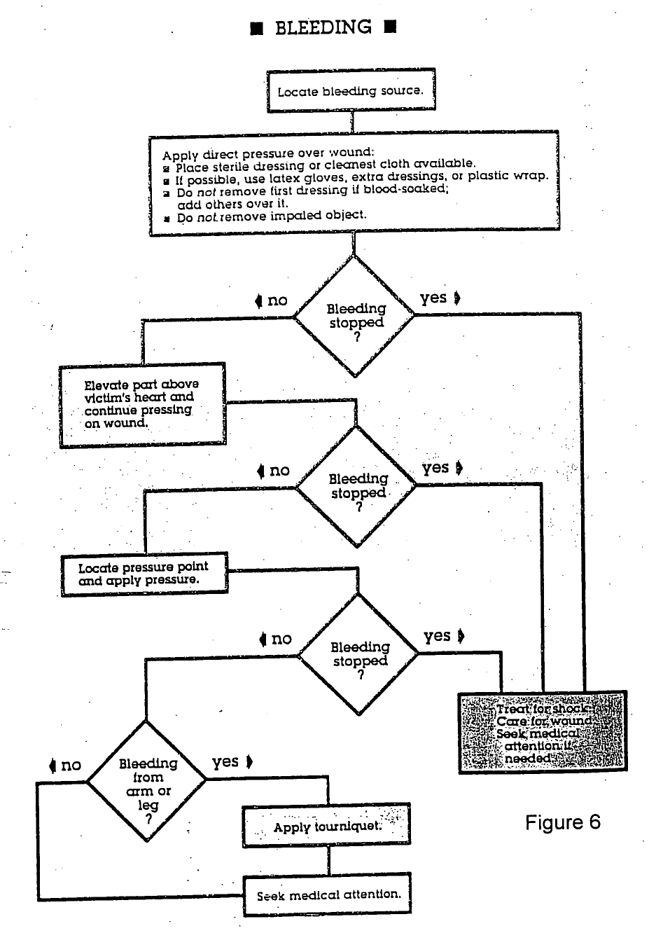

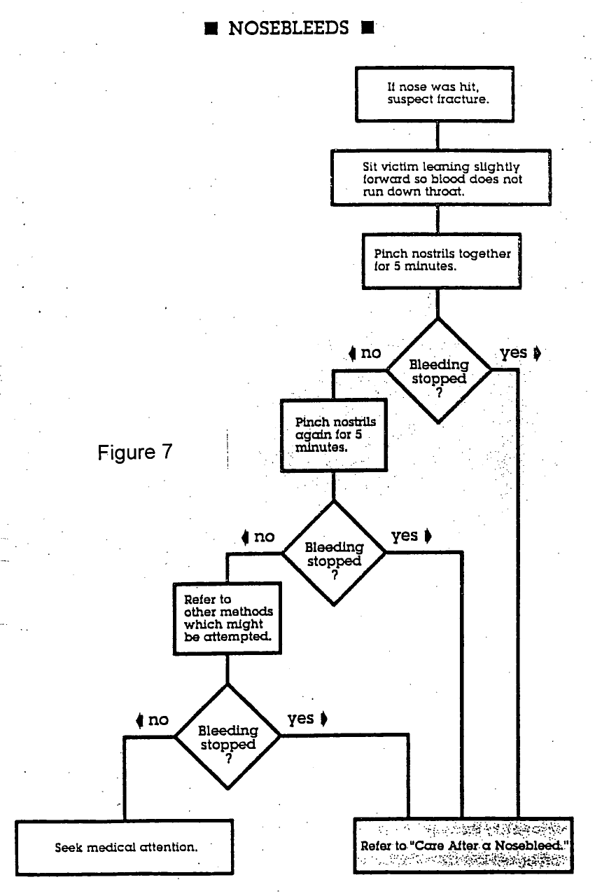

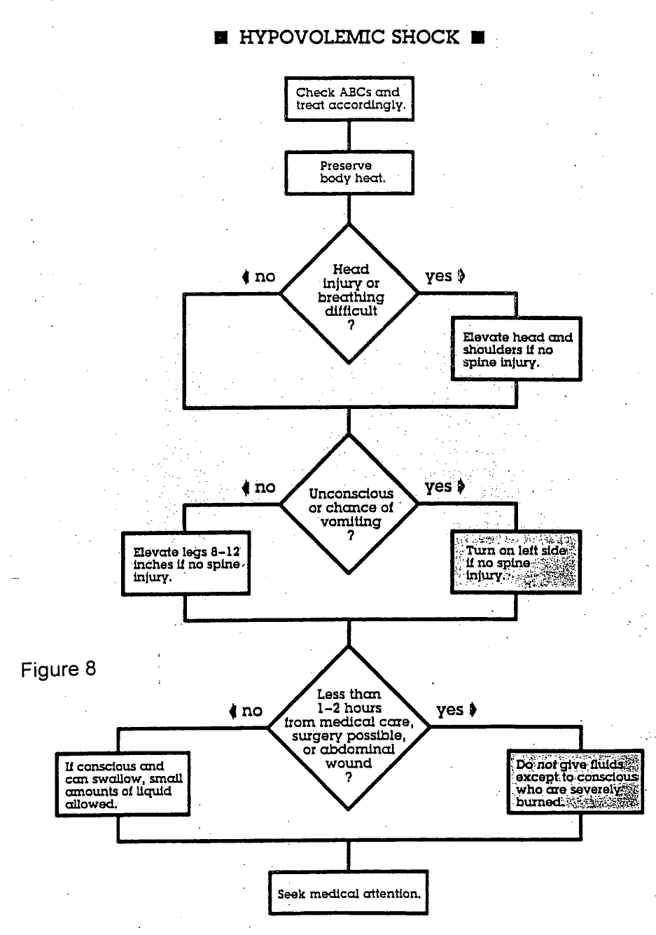

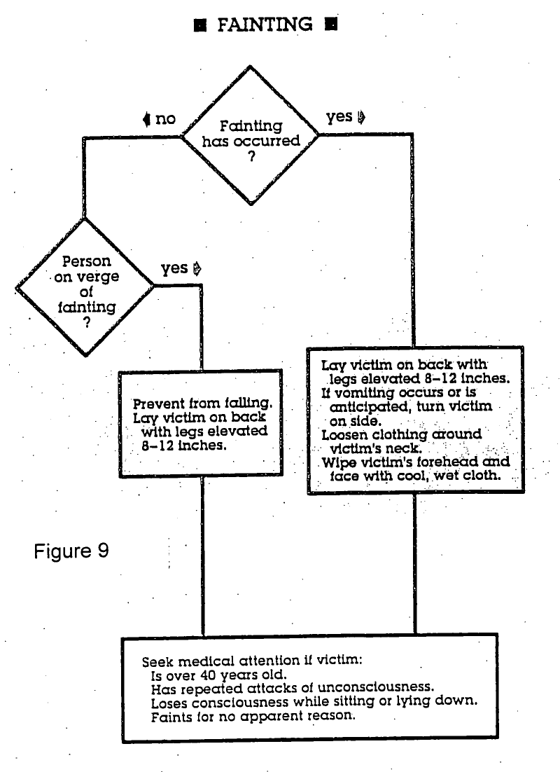

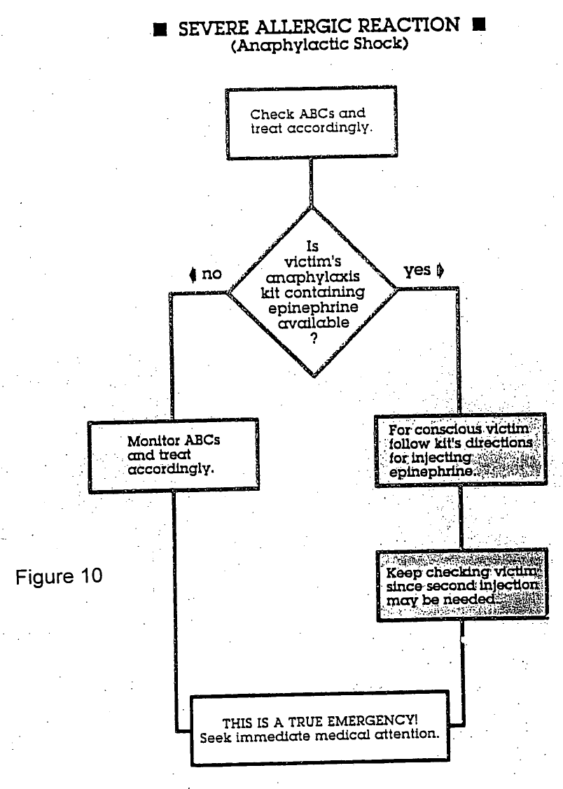

FIG. 6 illustrates an interactive menu of instructions for treating bleeding. FIG. 7 illustrates an interactive menu of instructions for treating nosebleeds. FIG. 8 illustrates an interactive menu of instructions for treating hypervolemic shock. FIG. 9 illustrates an interactive menu of instructions for treating fainting, and FIG. 10 illustrates an interactive menu of instructions for treating severe allergic reactions such as anaphylactic shock.

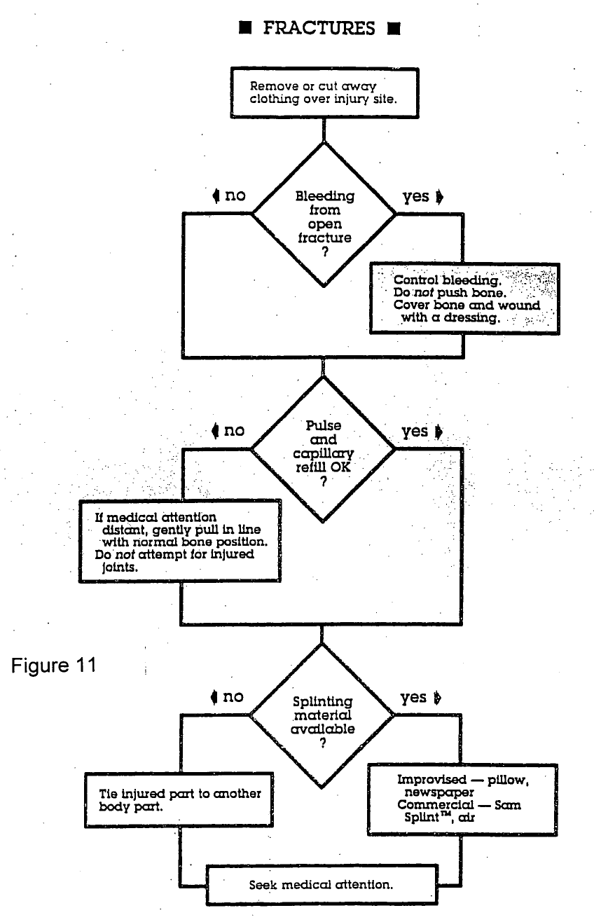

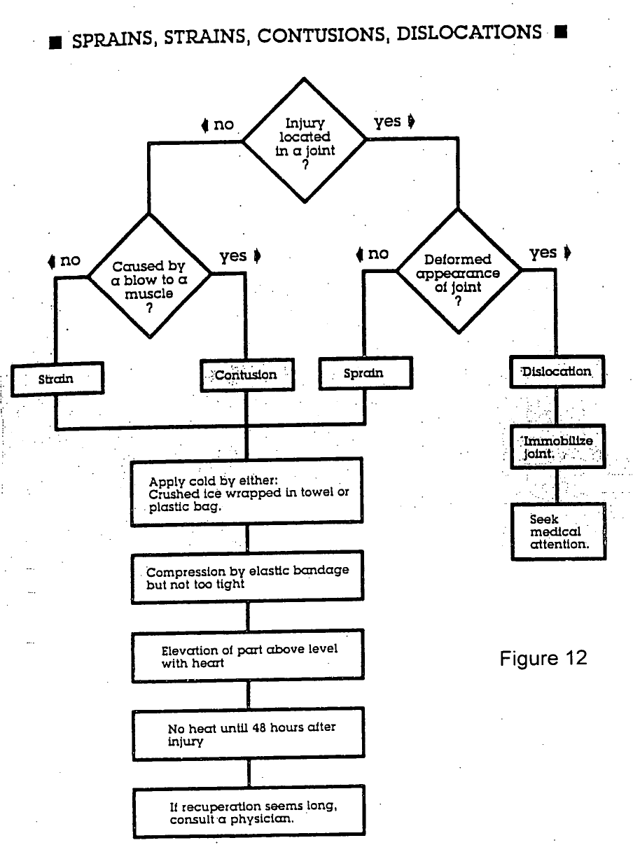

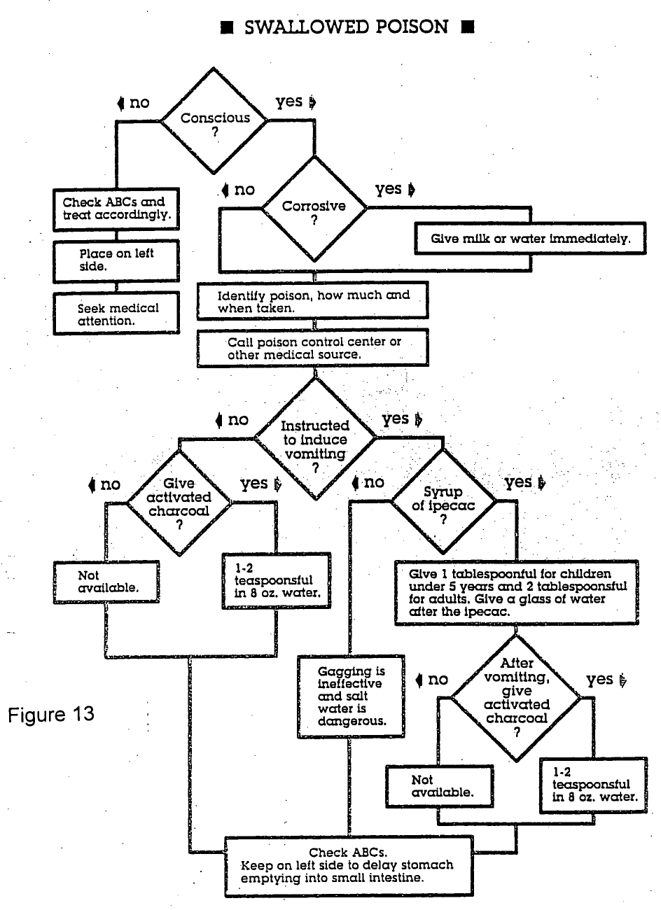

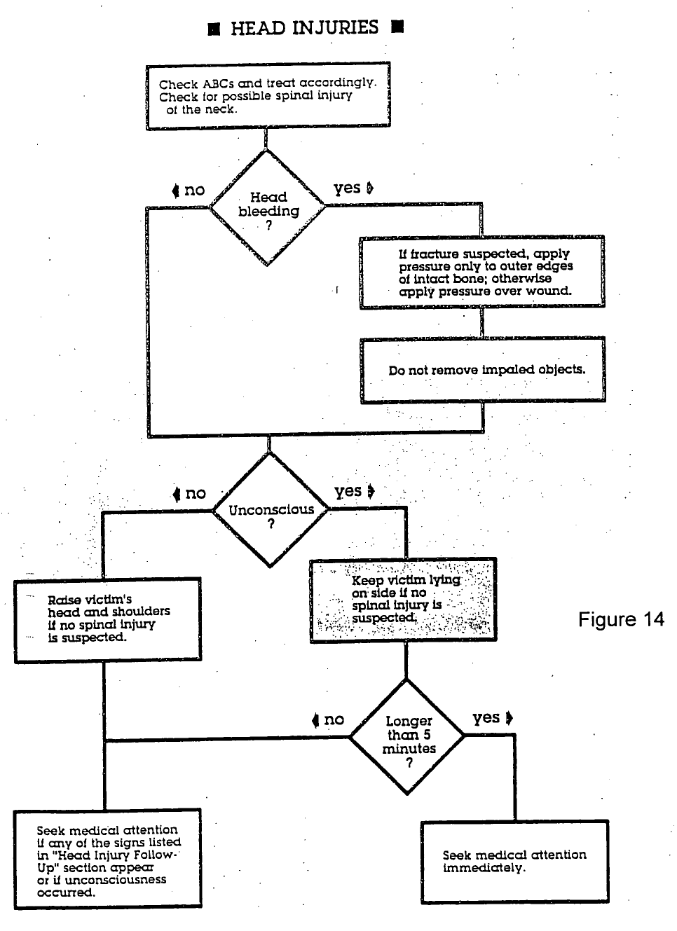

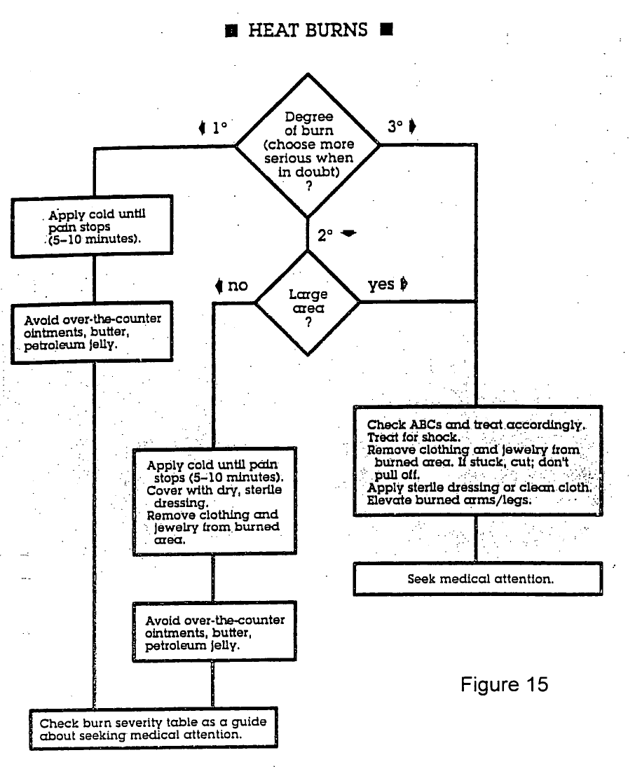

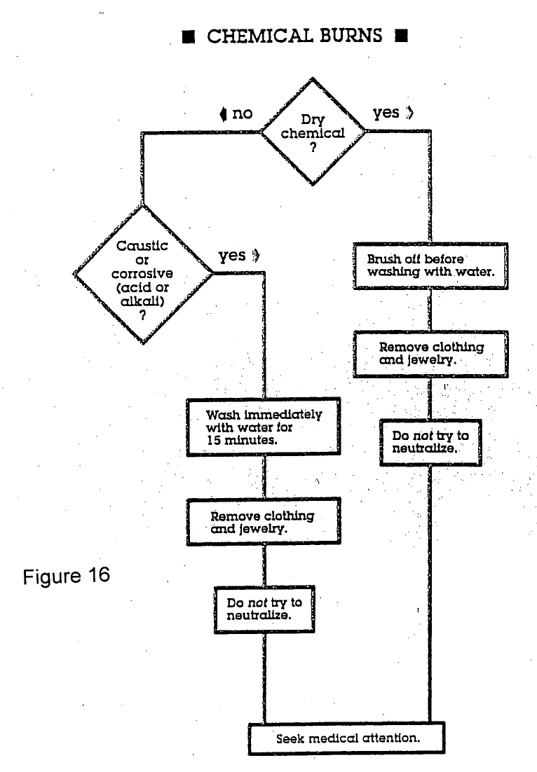

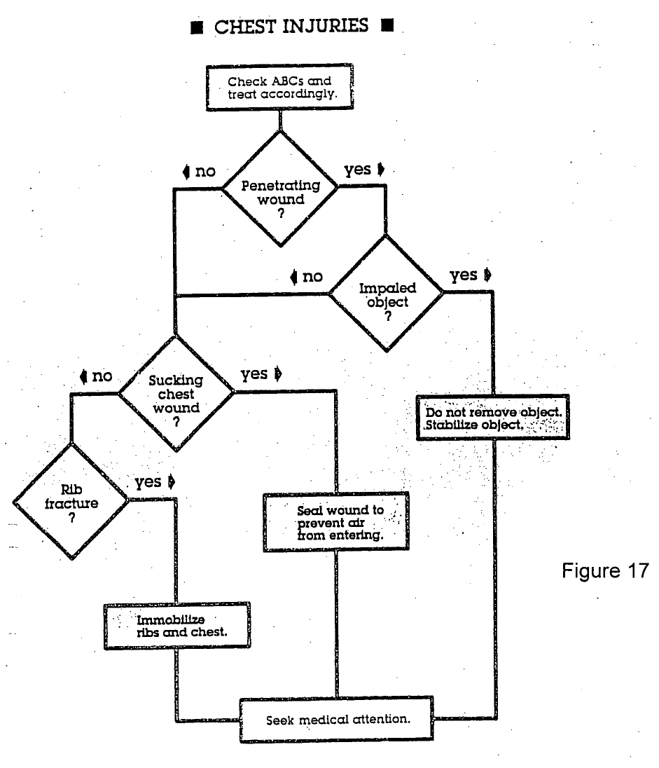

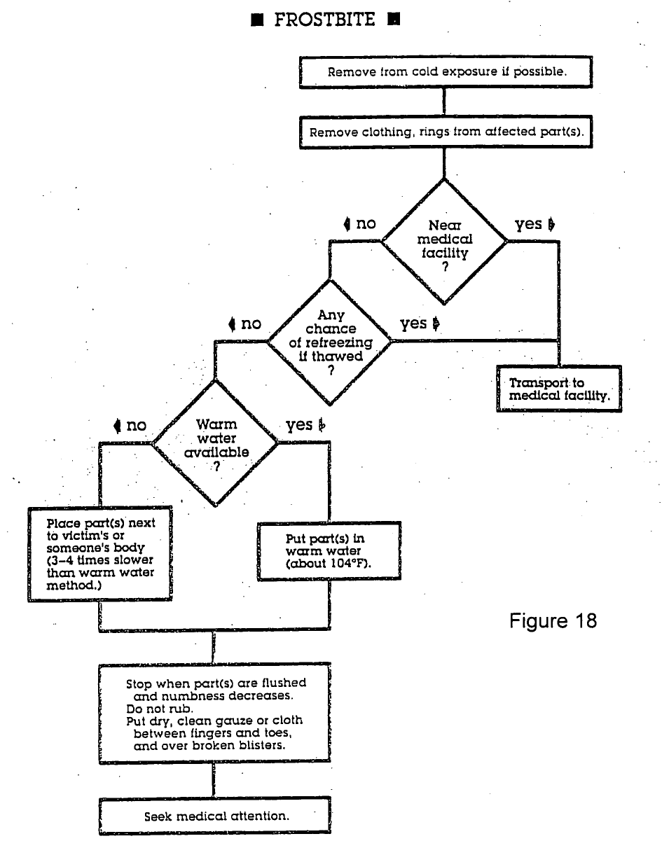

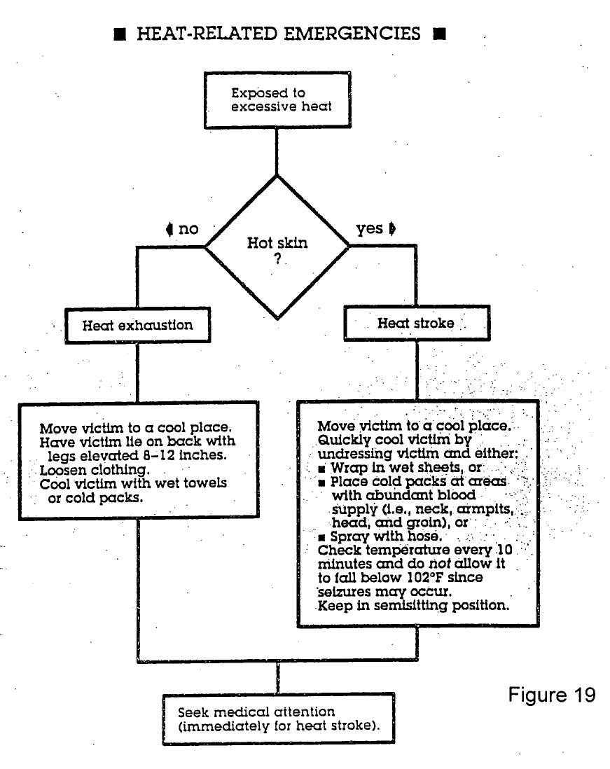

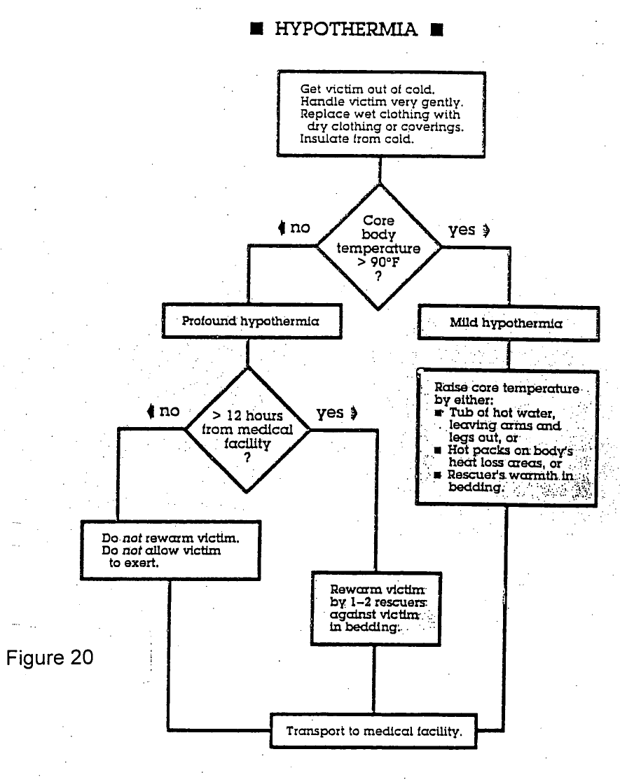

FIGS. 11 and 12 illustrate interactive menus of instructions for treating fractures, sprains, strains, contusions, and dislocations. FIG. 13 illustrates an interactive menu of instructions for treating swallowed poison. FIG. 14 illustrates an interactive menu of instructions for treating head injuries. FIG. 15 illustrates an interactive menu of instructions for treating heat burns, while FIG. 16 illustrates an interactive menu of instructions for treating chemical burns. FIG. 17 illustrates an interactive menu of instructions for treating a chest injury. FIG. 18 illustrates an interactive menu of instructions for treating frostbite. FIG. 19 illustrates an interactive menu of instructions to treat heat-related emergencies, and finally, FIG. 20 illustrates an interactive menu of instructions on treating hyperthermia.

Instructions for treating other medical emergencies, conditions, or situations, are also contemplated by the present invention, for example, treating electrocution, spinal injuries, animal bites, drowning, and the like. By way of example, an illustration showing how the present invention can be used to treat a chest injury will be discussed.

Upon finding an accident victim with a chest injury, the user would activate the present invention and access the interactive menus, regardless of the user's proximity to a wireless network. Upon activation, either by a dedicated sequence of the keyboard, voice activation, or other means known in the art, the menu would begin by asking the user to check the airwaves, the breathing, and the victim's cardio activity. The user would indicate completion of that task, whereupon the interactive menu would ask the user if the wound was a penetrating wound. Assuming the answer is no, the user would select no. Upon selection of the no option, another questions would appear, the question “is the wound a sucking chest wound?” The user could then inspect the accident victim and identify if the wound was a sucking chest wound, and, assuming here that it is a sucking chest wound, would select the answer yes, whereupon instructions to seal the wound and prevent air from entering the chest cavity would appear. Upon completion of that task, the final instructions to seek medical attention would be provided.

The present invention provides detailed instructions on an endless number of emergency medical ailments or other practical, needed instructions, and thus allows a user to provide medical assistance or other self-help, despite not having adequate training and no ability to use the device to contact another.

What is claimed is:

1. An apparatus providing emergency instructions comprising:
an electronic device with visual display; and
emergency instructions electronically resident in the device.

2. The apparatus of claim 1 further comprising:
an input unit for selecting from a menu of emergency instructions.

3. The apparatus of claim 1 further comprising:
an output unit for communicating the emergency instructions to a user.

4. An apparatus of claim 1 wherein the emergency instructions become resident in the device by storing the instructions in the device upon initial manufacture.

5. An apparatus of claim 1 wherein the emergency instructions become resident in the device by downloading the instructions into the device after manufacture.

6. The communication device of claim 1 wherein the device is a cell phone, a pocket personal computer, a pager, a walkie-talkie, a global positioning system, a cordless telephone, a kiosk display, or personal digital assistant.

7. The communication device of claim 2 wherein the input unit may receive input signals from at least one of a plurality of sources comprising buttons, voice recognition program, stylus, keyboard, touch screen, and microphone.

8. The communication device of claim 1 wherein the instructions comprise an interactive menu of instructions for treating a medical emergency.

9. The apparatus of claim 2 wherein the instructions are activated by a dedicated procedure.

10. An apparatus providing emergency instructions comprising:
an interactive computer program to communicate emergency instructions to a user;
an electronic device in which the computer program is stored; and
a visual display on the electronic device for displaying the emergency instructions.

11. The apparatus of claim 10 further comprising:
an input unit for selecting from a menu of emergency instructions.

12. The apparatus of claim 10 further comprising:
an output unit for communicating the emergency instructions to a user.

13. An apparatus of claim 10 wherein the emergency instructions become resident in the device by storing the instructions in the device upon initial manufacture.

14. An apparatus of claim 10 wherein the emergency instructions become resident in the device by downloading the instructions into the device after manufacture.

15. The communication device of claim 10 wherein the device is a cell phone, a pocket personal computer, a pager, a walkie-talkie, a global positioning system, a cordless telephone, a kiosk display, or personal digital assistant.

16. The communication device of claim 11 wherein the input unit may receive input signals from at least one of a plurality of sources comprising buttons, voice recognition program, stylus, keyboard, touch screen, and microphone.

17. The communication device of claim 10 wherein the instructions comprise an interactive menu of instructions for treating a medical emergency.

18. The apparatus of claim 11 wherein the instructions are activated by a dedicated procedure.

19. A method of displaying emergency instruction using a wireless communication device comprising:
providing a wireless communication device;
downloading at least one emergency instruction into the device;
storing the instruction in a computer readable media;
activating the instruction independent of the communication device's proximity to a zone or network of wireless communication;
selecting from menu options to identify proper treatment of a victim in an emergency; and
displaying emergency medical instruction.

20. The method of claim 19 wherein a user accesses the instructions to aid in studying first aid and cardio pulmonary resuscitation techniques.
